(19) United States
(12) Patent Application Publication
(10) Pub. No.: US 2025/0229036 A1
(43) Pub. Date: Jul. 17, 2025

(54) Multi-Chamber Syringe with Pressure Valve for Sequential Delivery of Fluids and Methods of Use

(71) Applicant: Becton, Dickinson and Company, Franklin Lakes, NJ (US)

(72) Inventors: Rahul Malviya, Salmon Arm (CA); Divik Bhargava, Meerut (IN); Hemant Vilas Belsare, Pune (IN)

(21) Appl. No.: 18/853,866

(22) PCT Filed: Mar. 31, 2023
(86) PCT No.: PCT/US2023/017045

§ 371 Date: Oct. 3, 2024

Related U.S. Application Data
(62) Division of application No. 63/327,191, filed on Apr. 4, 2022.

(51) Int. Cl. A61M 5/315; A61M 5/178; A61M 5/19; A61M 5/31

(52) U.S. Cl. CPC A61M 5/31596; A61M 5/19; A61M 2005/1787; A61M 2005/3128; A61M 2005/31598

(57) ABSTRACT

A multi-chamber syringe for sequential expulsion of a first fluid followed by a second fluid includes a fluid port for expulsion of the first fluid and the second fluid. The syringe also includes a first stopper including a channel and a second stopper slidably positioned in a barrel of the syringe. A first chamber configured to contain the first fluid is between a distal end of the barrel and the first stopper, and a second chamber configured to contain the second fluid is between the first stopper and the second stopper. The syringe also includes a valve which transitions between (i) a closed position, where fluid flow through the channel is prevented, when a fluid pressure in the second chamber is below a predetermined opening pressure, and (ii) an open position when fluid pressure in the second chamber is greater than or equal to the predetermined opening pressure.

### CROSS-REFERENCE TO RELATED APPLICATION

The present application claims priority to U.S. Provisional Application Ser. No. 63/327,191, entitled “Multi-Chamber Syringe with Pressure Valve for Sequential Delivery of Fluids and Methods of Use”, filed Apr. 4, 2022, the entire disclosure of which is hereby incorporated by reference in its' entirety.

### BACKGROUND OF THE INVENTION

### Field of the Invention

The present disclosure relates generally to syringes for expelling multiple fluids and, in particular, to a syringe for sequential expulsion of a first fluid, such as a medical fluid, followed by a second fluid, such as a flush solution.

### Description of Related Art

Vascular access devices (VADs) are commonly used medical devices, which can include intravenous (IV) catheters, such as peripheral catheters or central venous catheters. If not properly maintained or if exposed to a non-sterile environment, the VADs can become contaminated, sealed with blood clots, and/or can spread infection. Many medical facilities implement sterile practices and protocols to ensure that VADs are used properly and do not become sealed or infected. These protocols often include sterilizing the VAD and flushing the catheter with a flush solution. Specifically, VAD standards of practice usually recommend flush procedures be performed after catheter placement, before fluid infusion, and before and after drug administration, blood sampling, transfusions, and/or administration of parenteral nutrition.

These flush procedures are intended to confirm catheter patency, avoid drug incompatibilities, ensure that the complete drug dose is administered to the patient, prevent thrombus formation, and minimize a risk of bloodstream infections caused by contamination of the VAD. Further, flushing can prevent build-up of deposits of blood, blood residue, and IV drugs within a catheter or other VAD device. Such build-up can cause partial or complete blockage of the fluid pathway in a catheter system, which may require a practitioner to perform certain expensive and potentially dangerous processes for purging the affected catheter or may require the practitioner to exchange the blocked catheter with a new catheter. Often, such blockages lead to interruptions in therapy, which may compromise patient care. The build-up of residue within a catheter may also increase infection risk.

During administration of a drug or another medical fluid, a catheter or VAD is usually flushed both before and after delivery of the fluid from a syringe or from another drug delivery device to the VAD. In some cases, catheters can be flushed using syringe assemblies that are pre-filled with various fluids (referred to herein as flush solutions). The size of the pre-filled syringe used to flush the catheter varies based, for example, on a size and length of the catheter. Typically, pre-filled syringes for flushing a catheter or IV line contain a volume of about 1 mL, 3 mL, 5 mL, or 10 mL of flush solution. Many different syringes commercially available from different manufacturers can be used for providing the flush solution through a catheter or IV line of a patient including, for example, the BD PosiFlush™ pre-filled saline syringe by Becton, Dickinson and Company.

In order to perform a flushing procedure, a practitioner connects the pre-filled flushing syringe to the VAD and advances a plunger rod into a barrel of the pre-filled syringe, which expels the flush solution through a nozzle or fluid port of the pre-filled syringe to the VAD. After flushing, the pre-filled syringe is disconnected from the VAD so that the practitioner can administer a dose of the medical fluid. The medical fluid is conventionally contained in a vial and is drawn into a separate syringe which, after being filled with the medical fluid, is connected to the VAD. The medical fluid is then injected to the VAD by advancing a plunger rod through the syringe containing the medical fluid. After the medical fluid is injected to the VAD, the syringe is disconnected from the VAD and a second pre-filled flushing syringe is used to flush the VAD. Flushing the VAD after administering the medical fluid confirms patency of the catheter and advances any remaining medical fluid in the catheter to the patient ensuring that a full dose of the medical fluid is delivered to the patient.

Connecting or disconnecting a syringe or another device from the VAD introduces portions of the VAD to an unsterile outside environment, which increases a possibility that the VAD will become contaminated and/or will be a source of a bloodstream infection. In order to decrease a risk of bloodstream infection and to ensure VADs are used and maintained correctly, standards of practice require disinfecting and cleaning any fluid ports or connectors of the VAD each time that a new syringe is connected to the VAD. Accordingly, reducing a number of syringes that must be connected to the VAD during a drug delivery procedure simplifies drug administration by reducing a number of times that portions of the VAD must be disinfected and cleaned. Reducing the number of syringes or other fluid delivery devices used during a procedure also reduces opportunities for portions of the VAD to become contaminated, thereby reducing the risk for bloodstream infections. The syringes and methods of the present disclosure provide simplified drug administration procedures by reducing the number of syringes used during such procedures, thereby reducing a risk of contamination and bloodstream infection.

### SUMMARY OF THE INVENTION

According to an aspect of the disclosure, a multi-chamber syringe for sequential expulsion of at least a first fluid followed by a second fluid includes a barrel having a proximal end, a distal end with a fluid port for expulsion of the first fluid and the second fluid from the barrel, and a sidewall extending between the proximal end and the distal end of the barrel. The syringe also includes a first stopper including at least one channel and a second stopper slidably positioned in the barrel. A first chamber configured to contain the first fluid is between the distal end of the barrel and the first stopper, and a second chamber configured to contain the second fluid is between the first stopper and the second stopper. The syringe also includes a valve which transitions between (i) a closed position, where fluid flow through the at least one channel of the first stopper is prevented, when a fluid pressure in the second chamber is below a predetermined opening pressure, and (ii) an open position when fluid pressure in the second chamber is greater than or equal to the predetermined opening pressure, thereby establishing fluid communication between the second chamber and the first chamber through the at least one channel of the first stopper.

According to another aspect of the disclosure, a pre-filled flushing syringe includes the previously described multi-chamber syringe and a predetermined volume of the second fluid disposed in the second chamber of the pre-filled syringe. The pre-filled syringe is provided with the first chamber fluidly isolated from the second chamber, thereby containing the predetermined volume of the second fluid within the pre-filled syringe.

According to another aspect of the disclosure, a method for sequential expulsion of fluids from the previously described multi-chamber syringe includes the following steps: moving a plunger rod fixedly connected to the second stopper of the syringe in a proximal direction, which moves the first stopper of the syringe in the proximal direction, to aspirate the first fluid into the first chamber of the syringe barrel; and, once a dose of the first fluid is in the first chamber, moving the plunger rod in the distal direction, as a single continuous stroke, thereby causing the first fluid followed by the second fluid to be expelled from the fluid port of the syringe barrel.

Non-limiting illustrative examples of embodiments of the present disclosure will now be described in the following numbered clauses.

Clause 1: A multi-chamber syringe for sequential expulsion of at least a first fluid followed by a second fluid, the syringe comprising: a barrel comprising a proximal end, a distal end comprising a fluid port for expulsion of the first fluid and the second fluid from the barrel, and a sidewall extending between the proximal end and the distal end of the barrel; a first stopper comprising at least one channel, the first stopper being slidably positioned in the barrel with a first chamber configured to contain the first fluid between the distal end of the barrel and the first stopper; a second stopper slidably positioned in the barrel proximal to the first stopper with a second chamber configured to contain the second fluid between the first stopper and the second stopper; and a valve which transitions between (i) a closed position, where fluid flow through the at least one channel of the first stopper is prevented, when a fluid pressure in the second chamber is below a predetermined opening pressure, and (ii) an open position when fluid pressure in the second chamber is greater than or equal to the predetermined opening pressure, thereby establishing fluid communication between the second chamber and the first chamber through the at least one channel of the first stopper.

Clause 2: The syringe of clause 1, wherein movement of the second stopper through the barrel in a proximal direction causes the first stopper to move in the proximal direction to aspirate the first fluid into the first chamber through the fluid port of the barrel.

Clause 3: The syringe of clause 1 or clause 2, wherein advancing the second stopper through the barrel causes the first stopper to move through the barrel to expel the first fluid from the first chamber through the fluid port of the barrel.

Clause 4: The syringe of any of clauses 1-3, with the first stopper in a distal-most position, movement of the second stopper towards the first stopper causes fluid pressure in the second chamber to increase towards the predetermined opening pressure of the valve.

Clause 5: The syringe of clause 4, wherein, once the valve opens, continued distal movement of the second stopper towards the first stopper moves the second fluid from the second chamber, through the at least one channel of the first stopper, the first chamber, and the fluid port of the barrel, thereby expelling the second fluid from the barrel.

Clause 6: The syringe of any of clauses 1-5, wherein the barrel comprises at least one of polyester, polycarbonate, polypropylene, polyethylene, polyethylene terephthalate, or acrylonitrile butadiene styrene.

Clause 7: The syringe of any of clauses 1-6, wherein the fluid port comprises a nozzle having a minimum cross-sectional area that is smaller than a cross-sectional area of an outlet of the at least one channel of the first stopper.

Clause 8: The syringe of clause 7, wherein the cross-sectional area of the outlet of the at least one channel of the first stopper is at least 3.0 mm², preferably between about 3.1 mm² and about 3.3 mm², and the minimum cross-sectional area of the nozzle is preferably less than 2.8 mm².

Clause 9: The syringe of any of clauses 1-8, wherein the first stopper and/or the second stopper comprise a thermoplastic elastomer, such as natural rubber (e.g., isoprene).

Clause 10: The syringe of any of clauses 1-9, wherein the first stopper and/or the second stopper comprise a proximal end, a distal end, an outer surface extending between the proximal end and the distal end, and at least one annular rib protruding from the outer surface that seals against an inner surface of the sidewall of the barrel.

Clause 11: The syringe of any of clauses 1-10, wherein the predetermined opening pressure of the valve is at least 30 psi.

Clause 12: The syringe of any of clauses 1-11, wherein the valve is biased to and initially provided in the closed position.

Clause 13: The syringe of any of clauses 1-12, wherein the valve comprises a single use valve that transitions from the closed position to the open position when the fluid pressure of the second chamber is equal to or greater than the opening pressure of the valve, and which, once open, does not return to the closed position.

Clause 14: The syringe of any of clauses 1-13, wherein the valve comprises a one way check valve comprising at least one of a ball valve, compression sealing valve, diaphragm valve, tilting disc valve, clapper valve, or duckbill valve.

Clause 15: The syringe of any of clauses 1-14, wherein the valve comprises a compressible seal that covers an inlet of the at least one channel, when the valve is in the closed position, and which compresses to uncover the inlet when the fluid pressure of the second chamber is equal to or greater than the predetermined opening pressure of the valve.

Clause 16: The syringe of any of clauses 1-15, wherein the first stopper comprises a cavity extending inwardly from a proximal surface of the first stopper, and wherein the valve is seated in the cavity.

Clause 17: The syringe of clause 16, wherein an inlet of the at least one channel extends through an inner surface of the cavity, and wherein an outlet of the at least one channel extends through a distal surface of the first stopper.

Clause 18: The syringe of clause 17, wherein the valve comprises a sealing portion positioned to cover the inlet of the at least one channel when the valve is closed, and a compressible portion, which compresses as the fluid pressure increases, causing the sealing portion of the valve to retract into the cavity away from the inlet of the at least one channel.

Clause 19: The syringe of clause 18, wherein the compressible portion comprises an elastomeric bellows comprising at least one radially extending flange sized to contact the inner surface of the cavity to support the valve.

Clause 20: The syringe of any of clauses 16-19, wherein the first stopper comprises a protruding lip extending into the cavity that engages the valve, when the valve is in the open position, preventing the valve from closing when the fluid pressure in the second chamber decreases below the opening pressure of the valve.

Clause 21: The syringe of any of clauses 16-20, wherein an outer surface of the valve and/or an inner surface of the cavity comprises a lubricant coating so that the valve slides through the cavity as the fluid pressure of the second chamber increases.

Clause 22: The syringe of clause 21, wherein the lubricant coating comprises Polytetrafluoroethylene (PTFE).

Clause 23: The syringe of any of clauses 16-22, further comprising an annular sleeve around at least a portion of the valve separating elastomeric portions of the valve from elastomeric portions of the first stopper.

Clause 24: The syringe of any of clauses 1-23, further comprising a plunger rod connected to the second stopper for moving the second stopper through the barrel.

Clause 25: The syringe of clause 24, wherein the syringe is configured such that the first fluid followed by the second fluid are expelled from the syringe barrel from a single continuous advancement of the plunger rod distally into the syringe barrel.

Clause 26: The syringe of clause 24 or clause 25, wherein the syringe is configured such that the first fluid followed by the second fluid are expelled from the syringe barrel by single-handed operation of the plunger rod.

Clause 27: The syringe of any of clauses 24-26, wherein the plunger rod comprises a proximal end comprising a press plate, a distal end comprising a threaded connector, and a body extending between the proximal end and the distal end.

Clause 28: The syringe of clause 27, wherein the second stopper comprises a threaded cavity that engages the threaded connector of the plunger rod, thereby fixing the plunger rod to the second stopper.

Clause 29: The syringe of any of clauses 24-28, wherein the stoppers are configured such that moving the plunger rod in the proximal direction or the distal direction moves both the second stopper and the first stopper.

Clause 30: The syringe of any of clauses 24-29, wherein the plunger rod is fixedly connected to the second stopper and the plunger rod and/or the second stopper are not connected to the first stopper.

Clause 31: The syringe of any of clauses 24-30, further comprising a removable plunger cap engaged between the plunger rod and the barrel for preventing movement of the plunger rod until the removable plunger cap is removed from the syringe.

Clause 32: A pre-filled flushing syringe, comprising: the syringe of any of clauses 1-31; and a predetermined volume of the second fluid disposed in the second chamber of the pre-filled syringe, wherein the pre-filled syringe is provided with the first chamber fluidly isolated from the second chamber, thereby containing the predetermined volume of the second fluid within the pre-filled syringe.

Clause 33: The pre-filled syringe of clause 32, wherein the second fluid comprises a saline and/or heparin flush solution.

Clause 34: The pre-filled syringe of clause 32 or clause 33, wherein the syringe further comprises a plunger rod connected to the second stopper for moving the second stopper and/or the first stopper through the barrel.

Clause 35: The pre-filled syringe of clause 34, further comprising a removable plunger cap engaged between the plunger rod and the barrel of the syringe for preventing movement of the plunger rod until the plunger cap is removed, thereby maintaining fluid isolation of the first chamber and the second chamber until the plunger cap is removed.

Clause 36: A method for sequential expulsion of fluids from the syringe of any of clauses 1-31, the method comprising: moving a plunger rod fixedly connected to the second stopper of the syringe in a proximal direction, which moves the first stopper of the syringe in the proximal direction, to aspirate the first fluid into the first chamber of the syringe barrel; and once a dose of the first fluid is in the first chamber, moving the plunger rod in the distal direction, as a single continuous stroke, thereby causing the first fluid followed by the second fluid to be expelled from the fluid port of the syringe barrel.

Clause 37: The method of clause 36, wherein the single continuous stroke of the plunger rod is performed as a single-handed movement.

Clause 38: The method of clause 36 or clause 37, wherein the syringe is provided partially prefilled containing a predetermined volume of the second fluid in the second chamber.

Clause 39: The method of any of clauses 36-38, wherein aspirating the first fluid into the first chamber comprises attaching a needle to the fluid port of the syringe, inserting the needle into a fluid container, and drawing the first fluid from the fluid container to the first chamber of the syringe.

Clause 40: The method of any of clauses 36-39, further comprising attaching at least one patient line to the fluid port of the syringe barrel prior to moving the plunger rod in the distal direction, wherein the fluid expelled from the syringe passes to the patient through the at least one patient line.

Clause 41: The method of any of clauses 36-40, wherein the first fluid comprises a therapeutic agent.

### BRIEF DESCRIPTION OF THE DRAWINGS

FIG. 1A is a perspective view of a multi-chamber syringe, according to an aspect of the present disclosure.

FIG. 1B is a perspective exploded view of the multi-chamber syringe of FIG. 1A.

FIG. 2A is a side view of the multi-chamber syringe of FIG. 1A in an initial or partially filled position.

FIG. 2B is a side view of the multi-chamber syringe of FIG. 1A in a filled positon.

FIG. 2C is a cross-sectional view of the multi-chamber syringe of FIG. 1A with a filled second chamber and an empty first chamber.

FIG. 2D is a side view of the multi-chamber syringe of FIG. 1A in a final position after expulsion of the first fluid and the second fluid from the syringe.

FIG. 3A is an enlarged side view showing a valve of the syringe of FIG. 1A in the closed position.

FIG. 3B is an enlarged side view showing the valve of the syringe of FIG. 1A in an open position.

FIG. 4A is a proximal perspective view of a first stopper of the syringe of FIG. 1A.

FIG. 4B is a cross-sectional view of the first stopper of FIG. 4A.

FIG. 5A is a side view of the valve of the syringe of FIG. 1A.

FIG. 5B is a cross-sectional view of the valve of FIG. 5A.

FIG. 6 is a flow chart showing a method for expulsion of a first fluid followed by a second fluid from a multi-chamber syringe, according to an aspect of the present disclosure.

FIGS. 7A and 7B are cross-sectional views showing a valve of another exemplary multi-chamber syringe, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. As used herein, the term “proximal” refers to a portion or end of a device, such as a syringe or catheter, which is grasped, manipulated, or used by a practitioner or another user. The term “distal” refers to an end or portion of the device that is farthest away from the portion of the device that is grasped, manipulated, or used by the practitioner. For example, the “proximal end” of a catheter or IV line refers to the end including a fluid port that is connected to a fluid container, such as an IV bag or syringe. The “distal end” of the catheter or IV line refers to the end that is connected to the patient. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to multi-chamber syringes 10, 210 that expel a first fluid F1 followed by a second fluid F2 from a syringe barrel through a fluid port or nozzle of the syringe 10, 210 to, for example, a vascular access device. The first fluid F1 can be a medical fluid, which, as used herein, can refer to a medication, total parenteral nutrient (TPN) liquid, or another therapeutic agent used for treatment of chronic or acute conditions, as are known in the art. Exemplary therapeutic agents can include, for example, drugs, chemicals, biological, or biochemical substances that, when delivered in a therapeutically effective amount to the patient, achieve a desired therapeutic effect. The second fluid F2 can be a flush solution, such as saline and/or a heparin lock flush solution. An example of a saline flush solution is 0.9% sodium chloride USP for injection. An example of a heparin lock flush solution is 0.9% sodium chloride with 100 USP units of heparin sodium per mL or 10 USP units of heparin sodium per mL. Other flush solutions, as are known in the art, may also be used with the syringes 10, 210 of the present disclosure. The syringes 10, 210 can also be used for sequential delivery of a first medication or therapeutic agent followed by a different second medication or therapeutic agent.

The syringes 10, 210 of the present disclosure allow a practitioner, such as a medical technician, nurse, physician assistant, physician, or other trained or untrained clinicians or medical caregivers, to administer, for example, a medication followed by a flush solution without needing to change syringes 10, 210 between delivery of the first fluid F1 and the second fluid F2. Further, the syringes 10, 210 of the present disclosure allow the practitioner to provide the sequential delivery of the first fluid F1 followed by the second fluid F2 through a single continuous advancement of a plunger rod of the syringes 10, 210.

As used herein, “single continuous advancement of a plunger rod” means that the practitioner is able to push the plunger rod in a distal direction, through a barrel of the syringe 10, 210, as a single continuous stroke to expel the first fluid F1 followed by the second fluid F2 from the syringe barrel. The practitioner does not need, for example, to disconnect a syringe or another device from the VAD between delivery of the first fluid and the second fluid. Further, using the syringes 10, 210 of the present disclosure, the practitioner does not need to perform any other action, such as twisting, rotating, or pulling on the plunger rod or pressing another component or mechanism of the syringe 10, 210, in order to perform the sequential delivery of the first fluid F1 and the second fluid F2. Accordingly, the fluids F1, F2 can be expelled from the syringe 10, 210 in sequence in response solely to the single continuous stroke of the plunger rod in the distal direction by the practitioner, which can be performed as a “single-handed” operation or movement (i.e., the practitioner can hold the syringe 10, 210 and press the plunger rod through the barrel with one hand). Accordingly, the syringes 10, 210 of the present disclosure simplify processes for administering the first fluid F1 followed by the second fluid F2 to a VAD and/or patient compared to conventional fluid delivery practices.

In some examples, the syringes 10, 210 are provided as a partially pre-filled syringe, where a chamber of the syringe 10, 210 is filled with a flush solution during manufacturing. The partially pre-filled syringe 10, 210 can include caps, clips, retainers, and/or other packaging to hold the plunger rod in place and to ensure that the flush solution does not leak from the partially pre-filled syringe 10, 210 at unexpected times, such as during transport.

The syringes 10, 210 of the present disclosure are also configured to allow the practitioner to aspirate a medical fluid into the syringe 10, 210 prior to fluid delivery to the patient. For example, the practitioner can insert a nozzle or needle of the syringe 10, 210 into a vial containing the first fluid F1 (e.g., the medical fluid) and then aspirate the medical fluid into a chamber of the syringe 10, 210 by moving the plunger rod of the syringe 10, 210 in a proximal direction. After the first fluid F1 is aspirated into the chamber of the syringe 10, 210, sequential delivery of the first fluid F1 followed by the second fluid F2 can occur by connecting the syringe 10, 210 to a fluid port of a VAD and then moving the plunger rod of the syringe 10, 210 in the distal direction, thereby expelling the first fluid F1 followed by the second fluid F2 from the syringe 10, 210 to the VAD.

By eliminating the need to clean or disinfect portions of the VAD between delivery of the first fluid F1 and the second fluid F2, the syringes 10, 210 of the present disclosure simplify the fluid administration procedure, providing substantial time savings compared to conventional fluid administration practices. The syringes 10, 210 of the present disclosure also reduce infection risk and allow for flushing of the VAD immediately following administration of the medical fluid, which may prevent drug occlusion in the VAD. Further, the syringes 10, 210 of the present disclosure may have low or zero dead space, meaning that nearly all or all medical fluid contained in a first chamber of the syringe 10, 210 is flushed from the syringe 10, 210 as the second fluid F2 (e.g., the flush solution) moves through the syringe 10, 210 to the VAD.

### Multi-Chamber Syringe for Sequential Drug Delivery

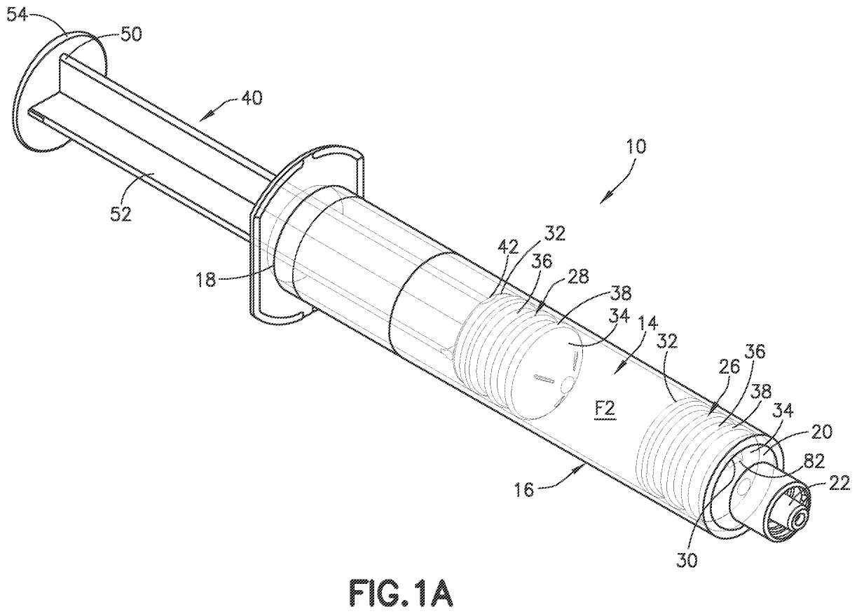

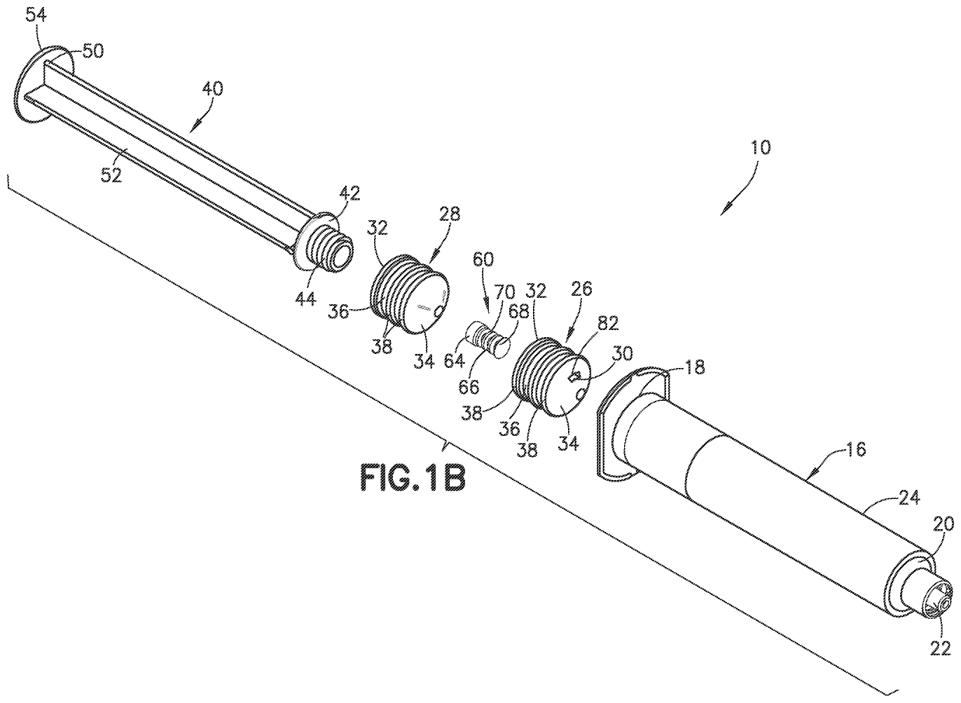

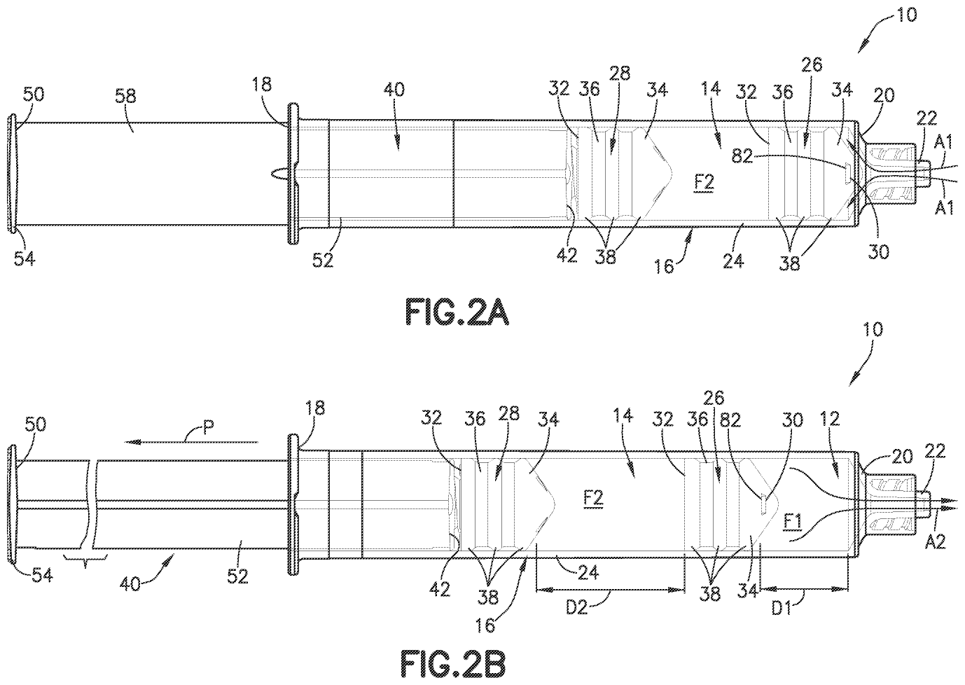

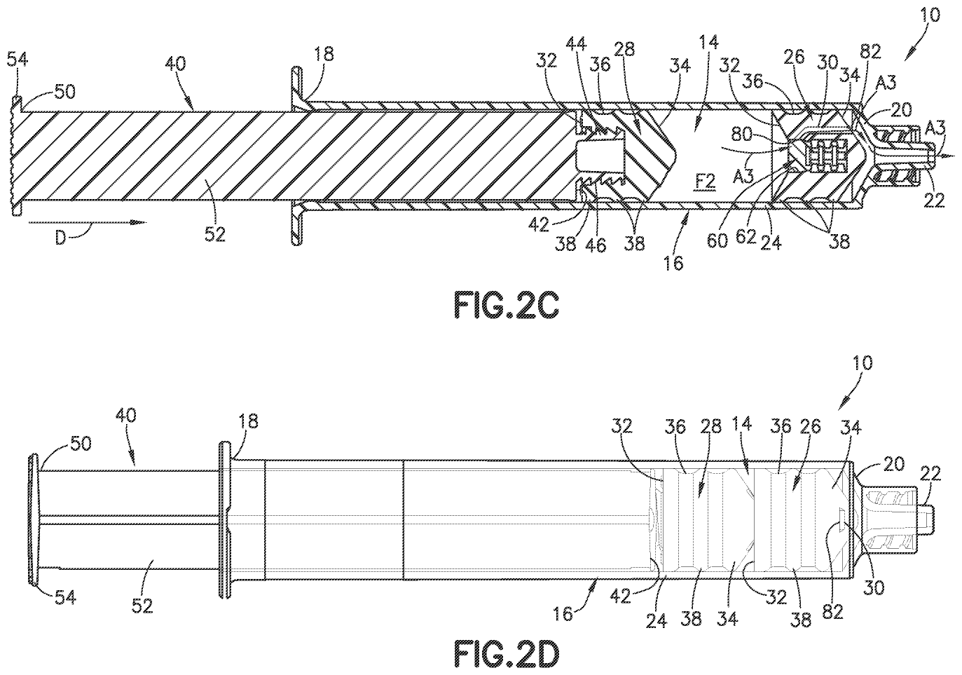

FIGS. 1A-2D illustrate an example of a multi-chamber syringe 10 for sequential expulsion of at least a first fluid F1 contained in the first chamber 12 followed by a second fluid F2 contained in a second fluid chamber 14. As previously described, the first fluid F1 can be a medical fluid, such as a drug or another therapeutic agent intended for delivery to a patient through a VAD, such as a catheter or IV line. The second fluid F2 can be a flush solution, such as saline solution and/or an anticoagulant, such as heparin. The type of flush solution and amount of flush solution contained in the second chamber 14 may vary depending, for example, on the specific type of catheter or IV line being used. In some examples, the syringe 10 contains or is configured to contain between about 1 mL and 20 mL of the second fluid F2 or, preferably, between about 5 mL and about 10 ml of the second fluid F2.

In some examples, the syringe 10 comprises a barrel 16 having an open proximal end 18, a distal end 20 including a nozzle or fluid port 22 for expulsion of the first fluid F1 and the second fluid F2 from the barrel 16, and a sidewall 24 extending between the proximal end 18 and the distal end 20 of the barrel 16. The fluid port 22 of the barrel 16 can be a connector, such as a luer connector, threaded connector, or snap connector, configured to be connected to a needle for accessing, for example, an interior of a medical vial containing a medical fluid. The fluid port 22 of the syringe 10 can also be configured to be connected directly or indirectly to a fluid port, valve, or another terminal access portion of a VAD. For example, a common type of fluid port of a VAD is a pierceable septum or pre-slit septum made of rubber or another elastomeric material, which permits insertion of a sharp or blunt needle cannula in order to infuse fluids or to withdraw fluids from a catheter of the VAD. Another common fluid port of a VAD is a valve, which does not require a needle for accessing the VAD. Instead, the valve can be activated by a frusto-conically shaped tip of the syringe barrel 16 to provide fluid communication between the interior of the barrel 16 and the VAD.

In some examples, the barrel 16 of the syringe 10 can be substantially similar in shape, size, and configuration to barrels of syringes used for administering a flush solution to a VAD, as are known in the art. For example, the barrel 16 can be a cylindrical structure formed from a rigid thermoplastic material, such as polyester, polycarbonate, polypropylene, polyethylene, polyethylene terephthalate, acrylonitrile butadiene styrene, or other injection moldable or formable resin materials, as are known in the art. Exemplary barrels for flush syringes are described, for example, in U.S. Patent Appl. Pub. No. 2020/0061297, entitled “Flush Syringe Assembly with Controlled Pulsatile Flushing,” which is incorporated herein by reference in its entirety.

The syringe 10 further comprises a distal or first stopper 26 and a proximal or second stopper 28 slidably positioned within the barrel 16 of the syringe 10. The stoppers 26, 28 separate the barrel 16 into the first chamber 12 and the second chamber 14. Specifically, as shown most clearly in FIG. 2B, the distal or first chamber 12 is between the distal end 20 of the barrel 16 and a distal end of the first stopper 26. The proximal or second chamber 14 is between a proximal end of the first stopper 26 and the second stopper 28.

The stoppers 26, 28 include many features of conventional syringe stoppers or plungers, as are known in the art. For example, the stoppers 26, 28 can be formed from a thermoplastic elastomer material, such as polypropylene or polyethylene, as well as from synthetic or natural rubber (e.g., isoprene). The first stopper 26 and/or the second stopper 28 can include a proximal surface 32 or proximal end, a distal surface 34 or end, an outer peripheral surface 36 extending between the proximal surface 32 and the distal surface 34. In some examples, the distal surface 34 or distal end of the stoppers 26, 28 can be conical or frusto-conical to assist in expelling fluid from the first chamber 12 of the barrel 16 through the fluid port 22. Alternatively, the distal end or distal surface 34 of the stoppers 26, 28 can be substantially flat, sloped, step-shaped, or other configurations depending, for example, on the size and shape of the barrel 16. The stoppers 26, 28 can further include one or more annular ribs 38 protruding from the outer peripheral surface 36. The annular ribs 38 are configured to seal against an inner surface of the sidewall 24 of the syringe barrel 16, ensuring that fluid moves through the syringe barrel 16 in an expected manner. In some examples, the stoppers 26, 28 include multiple annular ribs 38 in order to improve stability and to prevent the stoppers 26, 28 from tilting, shifting, or otherwise deforming as they move through the syringe barrel 16.

The first stopper 26 includes a channel 30 for allowing fluid to pass between the second chamber 14 and the first chamber 12. For example, the channel 30 can be an enclosed passage extending between the proximal surface 32 and the distal surface 34 of the first stopper 26. In other examples, the channel 30 can be a cutout portion of the stopper 26 extending radially inwardly from the peripheral outer surface 36 of the first stopper 26. As shown in FIGS. 1A-2D, the first stopper 26 includes one channel 30. In other examples, the first stopper 26 can include multiple channels for increasing a volume of fluid that can pass through the first stopper 26. For example, the first stopper 26 can be a vertically symmetrical (i.e., symmetrical through a vertical plane) structure having a first channel on one side of the stopper 26 and a symmetrical second channel on an opposite side of the first stopper 26.

The channel 30 can include an inflow portion or inlet 80, which receives the second fluid F2 from the second chamber 14, and an outflow portion or outlet 82 for expelling the second fluid F2 from the channel 30 into the first chamber 12. In some examples, the inlet 80 is an opening extending through the proximal surface 32 of the first stopper 26 and the outlet 82 is an opening extending through the distal surface 34 of the first stopper 26. The inlet 80, outlet 82, and channel 30 can have a variety of shapes and configurations. For example, as shown in FIG. 1B, the channel 30 and outlet 82 can have a rectangular cross-sectional shape. In other examples, the inlet 80, outlet 82, and/or channel 30 can have a square, circle, oval, or another regular cross-sectional shape.

Dimensions of the channel 30 can be selected in order to ensure that fluid flows through the channel 30 at a reasonable rate as the stoppers 26, 28 move through the barrel 16. If the channel 30 is too narrow, fluid flow rate through the channel 30 may increase by an unreasonable amount due to pneumatic forces or a Venturi effect. In some examples, a minimum cross-sectional area of the channel 30 is at least 3.0 mm², or preferably from about 3.1 mm² to about 3.3. mm². In some examples, a cross-sectional area of the fluid port 22 or nozzle of the barrel 16 is less than the cross-sectional area of the channel 30, which ensures that a volume of fluid entering the first chamber 12 through the channel 30 is greater than or equal to a volume of fluid being expelled from the syringe barrel 16 through the fluid port 22. For example, the fluid port 22 or nozzle of the barrel 16 can have a cross-sectional area of less than 2.8 mm², or preferably of about 2.77 mm².

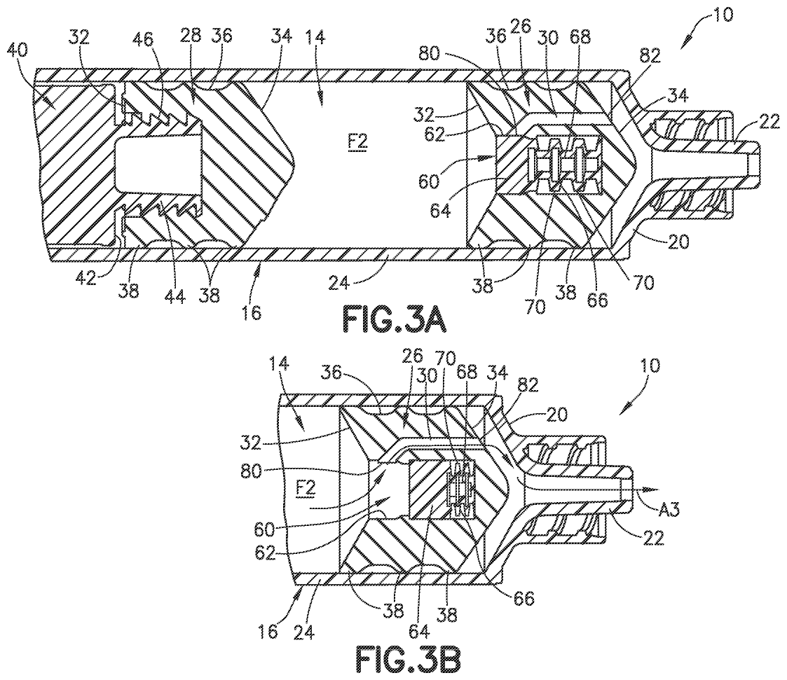

The syringe 10 further comprises a valve 60 for controlling fluid flow through the channel 30 of the first stopper 26. The valve 60 is configured to transition between a closed position (shown in FIG. 3A), where fluid flow through the channel 30 of the first stopper 26 is prevented, and an open position (shown in FIG. 3B), where fluid flow through the channel 30 occurs. The valve 60 can be a pressure-activated valve meaning that the valve 60 transitions between the closed position and the open position due to changes in fluid pressure in the second chamber 14 of the syringe barrel 16. In some examples, the valve 60 is initially provided in the closed position and remains in the closed position as long as the fluid pressure in the second chamber 14 is below a predetermined activation or opening pressure of the valve 60. When the fluid pressure in the second chamber 14 increases, becoming equal to or greater than the activation or opening pressure, the valve 60 transitions to the open position.

The activation or opening pressure can be selected based on fluid pressures that commonly occur when a stopper is manually moved through a barrel of a flushing syringe at a reasonable rate, as occurs when a practitioner pushes a plunger rod of a flushing syringe through the syringe barrel. In some examples, the activation or opening pressure for the valve 60 is at least 30 psi, though the activation or opening pressure can be selected or optimized for different syringe designs taking into account, for example, the size, shape, and materials of the stoppers 26, 28, syringe barrel 16, and other components of the syringe 10. In some examples, the syringe 10 is configured such that the activation or opening pressure for the valve 60 is greater than a pressure required to infuse the drug (i.e., the pressure required to expel the first fluid F1 from the first chamber 12 through the fluid port 22 and to the patient through the VAD). Accordingly, once the first stopper 26 reaches a distal-most position within the barrel 16 (shown in FIG. 2C), the practitioner may be required to apply a greater force to a plunger rod of the syringe 10 connected to the second stopper 28 in order to activate or open the valve 60 than is required to move the stoppers 26, 28 through the barrel 16. Due to these differences in the force that must be applied to the plunger rod, the practitioner receives feedback (i.e., a feeling that increased force on the plunger rod is needed) indicating that the first fluid F1 has been expelled from the syringe 10 and that the first stopper 26 is in its distal-most position in the barrel 16.

The stoppers 26, 28 of the syringe 10 of the present disclosure are configured to move through the barrel 16 simultaneously and in a coordinated manner, even though the stoppers 26, 28 are not mechanically connected or engaged together. This simultaneous movement occurs due to the incompressible property of a liquid, such as the second fluid F2, in the second chamber 14 of the barrel 16. More specifically, pressure applied to the distal or second stopper 28 is transferred to a fluid column of the second fluid F2 (e.g., the flush solution) in the second chamber 14. This fluid column applies pressure both to the proximal or first stopper 26 and to the valve 60 causing the first stopper 26 and valve 60 to move through the barrel 16 toward the distal-most position (shown in FIG. 2C), thereby expelling the first fluid F1 from the first chamber 12 of the syringe barrel 16. When the first stopper 12 is in the distal-most position, the distal end 20 of the barrel 16 prevents further movement of the first stopper 26, meaning that continuing to apply pressure to the second stopper 28 causes the fluid pressure in the second chamber 14 to increase. The increase in fluid pressure causes the valve 60 to open, as previously described, thereby allowing the second fluid F2 to be expelled from the syringe 10 through the channel 30 and the fluid port 22 of the barrel 16.

In a similar manner, the stoppers 26, 28 also move together through the barrel 16 in the proximal direction even though the stoppers 26, 28 are not mechanically connected or engaged together. Specifically, moving the second stopper 28 in the proximal direction produces a vacuum or negative pressure in the syringe barrel 16, which draws the proximal or first stopper 26 through the barrel 16 in the proximal direction along with the second stopper 28. Accordingly, movement of the second stopper 28 through the barrel 16 in a proximal direction, as shown by arrow P in FIG. 2B, aspirates the first fluid F1 into the first chamber 12 through the fluid port 22 of the barrel 16 in a similar manner as occurs when aspirating a fluid from a vial into a conventional syringe.

The stoppers 26, 28 are configured to move between several positions or configurations during a fluid expulsion procedure. The syringe 10 may initially be provided in a partially-filled configuration, as shown in FIG. 2A, with a second fluid F2, such as flush solution, in the second chamber 14. In this initial or partially-filled position, the stoppers 26, 28 are spaced apart from one another by a distance D1 (shown in FIG. 2B) sufficient to contain a volume of the second fluid F2 in the second chamber 14 of about 1 mL to 20 mL, or preferably about 5 mL to 10 mL. When ready for use, if the first stopper 26 is not in the distal-most position within the barrel 16, the practitioner can move the first stopper 26 distally through the barrel 16 to seat the first stopper 26 against the distal end 20 of the barrel 16. The practitioner can then move the stoppers 26, 28 in the proximal direction (shown by arrow P in FIG. 2B) to aspirate the first fluid F1 into the first chamber 12, thereby fully filling the syringe 10, as shown in FIG. 2B.

As shown in FIG. 2B, when the syringe 10 is fully filled, the first stopper 26 is a distance D2 from the distal end 20 of the barrel 16. Once the syringe 10 is fully filled, moving the stoppers 26, 28 in the distal direction (shown by arrow D in FIG. 2C) moves the first stopper 26 towards the distal end 20 of the barrel 16, thereby expelling the first fluid F1 from the first chamber 12. The syringe 10 is shown in an intermediate position with the full second chamber 14 and the empty first chamber 12 in FIG. 2C. Continued distal movement of the stoppers 26, 28 causes the fluid pressure in the second chamber 14 to increase towards the activation or opening pressure of the valve 60. Once the fluid pressure in the second chamber 14 exceeds the activation or opening pressure of the valve 60, the valve 60 opens thereby establishing fluid communication between the second chamber 14 and the first chamber 12 through the channel 30. Once fluid communication between the chambers 12, 14 is established, the second fluid F2 is then expelled from the syringe 10 by moving the second stopper 28 towards the first stopper 26. Specifically, movement of the second stopper 28 towards the first stopper 26 causes the second fluid F2 to move from the second chamber 14 to the first chamber 12, and then from the first chamber 12 through the fluid port 22. FIG. 2D shows the syringe 10 in an end-of-use or final position after the fluids F1, F2 are expelled from the syringe barrel 16. In the end-of-use or final position, the distal surface 34 of the second stopper 28 is in contact with or nearly in contact with the proximal surface 32 of the first stopper 26.

With continued reference to FIGS. 1A-2D, the syringe 10 further comprises a plunger rod 40 connected to the second stopper 28 for moving the second stopper 28 through the syringe barrel 16. As previously described, moving the second stopper 28 through the barrel 16 causes the first stopper 26 to simultaneously move through the barrel 16 in coordination with the second stopper 28, even though the stoppers 26, 28 are not mechanically connected or engaged together. The plunger rod 40 can be, for example, an injection molded part formed from a rigid thermoplastic material, such as polyester, polycarbonate, polypropylene, polyethylene, polyethylene terephthalate, or another thermoplastic material, as are known in the art. The plunger rod 40 can be a conventional plunger rod used in currently available syringes, as are known in the art, and can be connected to the second stopper 28 by standard mechanical connectors, fasteners, or adhesives. In other examples, the plunger rod 40 can be integrally formed or co-molded with the second stopper 28.

In some examples, the plunger rod 40 includes a distal end 42 engaged to the second stopper 28. For example, as most clearly seen in FIGS. 1B and 2C, the distal end 42 of the plunger rod 40 can include a threaded connector 44 that is inserted into a corresponding cavity 46 (shown in FIG. 2C) extending inwardly from the proximal surface 32 of the second stopper 28. The plunger rod 40 also includes a proximal end 50 protruding proximally from the proximal end 18 of the syringe barrel 16, and a body 52 extending between the proximal end 50 and the distal end 42 of the plunger rod 40. The proximal end 50 of the plunger rod 40 can include a thumb press plate 54 for manipulating the plunger rod 40 to move the stoppers 26, 28 through the syringe barrel 16. The body 52 of the plunger rod 40 can have a variety of cross-sectional shapes and configurations within the scope of the present disclosure. For example, the body 52 can have a generally cross shaped cross-section. In other examples, the cross-section of the plunger rod 40 can be an I-beam shape, circle, square, L-shaped, or other conventional shapes that can be formed by injection molding processes, as are known in the art. In some examples, the syringe 10 can also include a removable plunger cap 58 (shown in FIG. 2A), such as a partially cylindrical (i.e., a cylindrical body with an axial slot for removing the cap 58 from the syringe 10) or c-shaped spacer, engaged between the thumb press plate 54 of the plunger rod 40 and the proximal end 18 of the barrel 16 for preventing movement of the plunger rod 40 until the removable plunger cap 58 is removed from the syringe 10. The plunger rod cap 58 prevents the plunger rod 40 from being pressed into the syringe barrel 16 at inappropriate times, such as during transport of a pre-filled syringe from a manufacturing facility to a customer.

As previously described, the valve 60 controls fluid flow between the second chamber 14 and the first chamber 12 through the channel 30 of the first stopper 26. Structures of the valve 60, channel 30, and first stopper 26 that provide fluid communication between the chambers 12, 14 of the syringe 10 at appropriate times to allow for sequential delivery of the first fluid F1 followed by the second fluid F2 will now be described in further detail, with specific reference to FIGS. 3A-5B. It is understood, however, that many different valve structures can be used with the syringe 10 of the present disclosure. Accordingly, the valve structures described herein are intended to provide non-limiting examples of types of pressure-activated fluid valves that can be used with the syringe barrel 16 and stoppers 26, 28 of the present disclosure.

In some examples, the pressure-activated valve 60 is a one-way check valve. As used herein, a one-way check valve (also referred to as a non-return valve, retention valve, or one-way valve) refers to a valve that allows a flow of fluid through the valve in only one direction. For the syringe 10, the valve 60 is configured to permit fluid flow from the second chamber 14 to the first chamber 12. Fluid flow from the first chamber 12 to the second chamber 14 is prevented. The one-way check valve 60 can be, for example, a ball valve, diaphragm valve, tilting disc valve, clapper valve, or duckbill valve, as are known in the art.

In some examples, the valve 60 is a single use valve. As used herein, a “single-use” valve refers to a valve that is initially provided in a closed position and transitions to an open position one time. The single-use valve does not return to the closed position after being opened. For example, the valve 60 can be configured to transition from the closed position to the open position when the fluid pressure of the second chamber 14 is equal to or greater than the activation or opening pressure of the valve 60. Once the valve 60 opens, the valve 60 does not return to the closed position. An advantage of a single-use valve is that the fluid pressure of the second chamber 14 does not need to remain above the activation or opening pressure of the valve 60 for the entire time that the second fluid F2 is being pushed through the channel 30 of the first stopper 26. Instead, the practitioner need only press the plunger rod 40 with sufficient force to create a fluid pressure sufficient to open the valve 60. Once the valve 60 is open, the practitioner can push the plunger rod 40 with less force to expel the second fluid F2 from the second chamber 14 through the channel 30 because the valve 60 does not return to the closed position even when the fluid pressure in the second chamber 14 drops below the activation or opening pressure of the valve 60.

With reference to FIGS. 3A and 3B, in some examples, the valve 60 is a compressible sealing valve positioned to cover the inlet 80 of the channel 30 of the first stopper 26 when the valve 60 is in the closed position (shown in FIG. 3A). The valve 60 can be seated in a cavity 62 extending inwardly from the proximal surface 32 of the first stopper 26, with the inlet 80 of the channel 30 extending through an inner surface of the cavity 62. The valve 60 is configured to compress to uncover the inlet 80 when the fluid pressure of the second chamber 14 is equal to or greater than the predetermined activation or opening pressure of the valve 60. The valve 60 is shown in the open position in FIG. 3B. More specifically, the valve 60 can include a sealing portion 64 positioned to cover the inlet 80 when the valve 60 is closed and a compressible portion 66 that compresses as the fluid pressure increases. The sealing portion 64 and the compressible portion 66 can be integrally formed by, for example, an injection molding process, as is known in the art. In other examples, the sealing portion 64 and the compressible portion 66 can be separate structures, such as components made by separate molding processes, which are connected together by, for example, a mechanical fastener or adhesive. As previously described, the sealing portion 64 and/or compressible portion 66 can be formed from a thermoplastic elastomer, such as polypropylene or polyethylene, as well as from synthetic or natural rubber (e.g., isoprene).

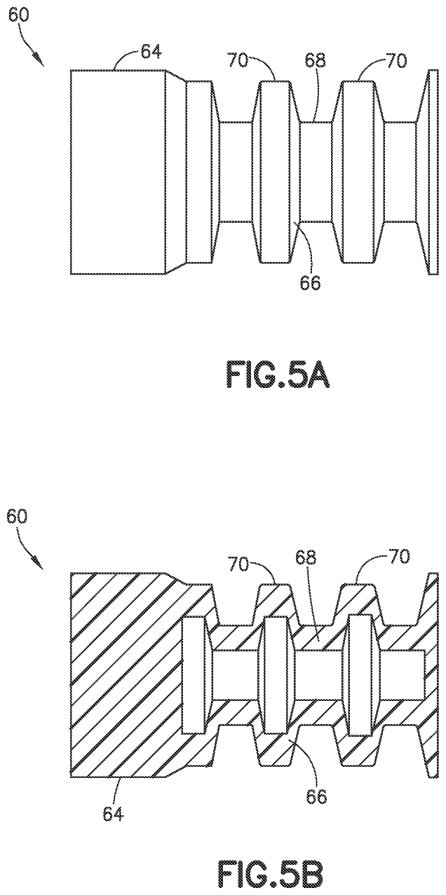

As shown in FIGS. 3A, 3B, 5A, and 5B, the sealing portion 64 is a cylindrical structure having an outer diameter that substantially corresponds to an inner diameter of the cavity 62. Further, the sealing portion 64 can include a substantially vertical annular peripheral surface in contact with a vertical wall of the cavity 62 and which covers the inlet 80 of the channel 30 when the valve 60 is closed, preventing fluid in the second chamber 14 from entering the channel 30. The compressible portion 66 can include a variety of different structures configured to axially compress when an axial force is applied thereto, thereby reducing a height of the compressible portion 66 and causing the sealing portion 64 to retract into the cavity 62. For example, the compressible portion 66 can be formed from compressible material(s), such as a porous material and/or foam, which decreases in volume when an external force is applied thereto. In other examples, the compressible portion 66 can be formed from flexible materials that bend, deform, collapse, or otherwise change position when an external force is applied thereto, thereby decreasing the axial height of the compressible portion 66.

In some examples, as shown in FIGS. 3A, 3B, 5A, and 5B, the compressible portion 66 can be a hollow elastomeric bellows extending distally from the sealing portion 64. The elastomeric bellows comprises an elongated hollow central portion 68 and radially extending flanges 70 that extend radially outwardly from the central portion 68. When an axial force is applied to the valve 60, the central portion 68 of the bellows flexes or bulges outwardly, thereby reducing the axial height of the compressible portion 66 and causing the sealing portion 64 to move away from the inlet 80 to allow fluid flow into the channel 30. In some examples, the flanges 70 can be sized to press against and/or contact the inner surface of the cavity 62 to support the valve 60. For example, contact between the flanges 70 and inner surface of the cavity 62 can maintain the valve 60 in an upright position and prevent the valve 60 from tilting or falling over.

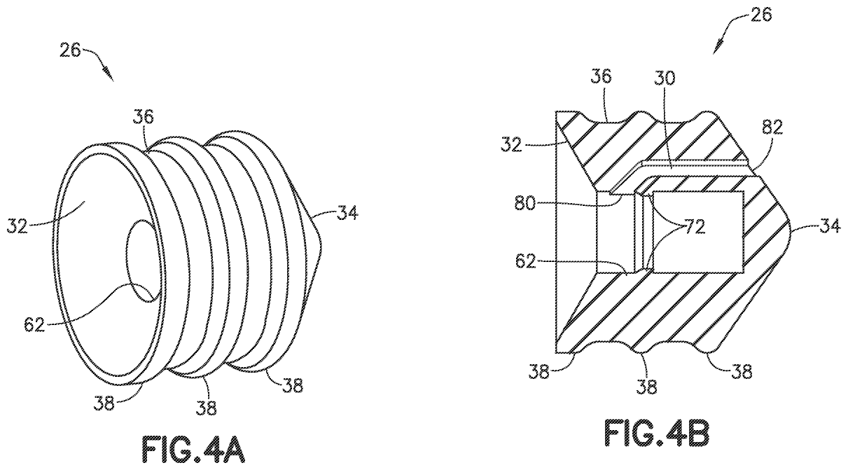

As previously described, the valve 60 can be a single use valve. In such cases, in order to prevent the valve 60 from closing once opened, the first stopper 26 can include one or more protrusions, baffles, or other blocking surfaces extending from an inner surface of the cavity 62 for maintaining the valve 60 in the open or compressed position. For example, as shown in FIGS. 4A and 4B, the first stopper 26 can include a protruding lip 72 (shown in FIG. 4B) extending into the cavity 62 that engages the valve 60, when the valve 60 is in the open position (shown in FIG. 3B). For example, as the valve 60 closes, the sealing portion 64 can deform, retracting into the cavity 62 past the protruding lip 72. When the valve 60 is in the open positon, the protruding lip 72 can contact and engage a proximal surface of the sealing portion 64 to prevent the compressible portion 66 and/or sealing portion 64 from expanding through the cavity 62. Accordingly, the engagement between the sealing portion 64 and the protruding lip 72 prevents the valve 60 from closing and/or prevents the sealing portion 64 from covering the inlet 80 even after fluid pressure in the second chamber 14 decreases below the activation or opening pressure of the valve 60.

As is known in the art, parts formed from elastomeric materials, such as natural rubber (e.g., isoprene), may stick together due to stationary friction (e.g., stiction). For the syringe 10, stiction between the first stopper 26 and the valve 60 may prevent the valve 60 from moving through the cavity 62 in an expected manner. This stiction may cause problems during assembly of the first stopper 26 and valve 60. In some cases, effects of stiction become more significant if the first stopper 26 and valve 60 are in contact for long periods of time, which can limit the shelf life of the syringe 10. In order to avoid stiction between the first stopper 26 and valve 60, the inner surface of the cavity 62 and/or valve 60 can be coated with a lubricant coating to promote sliding of the valve 60 through the cavity 62. Desirably, the lubricant material is a non-toxic medical grade lubricant suitable for use with medical devices. For example, the lubricant coating can be Polytetrafluoroethylene (PTFE).

### Method of Expelling Fluid From a Multi-Chamber Syringe

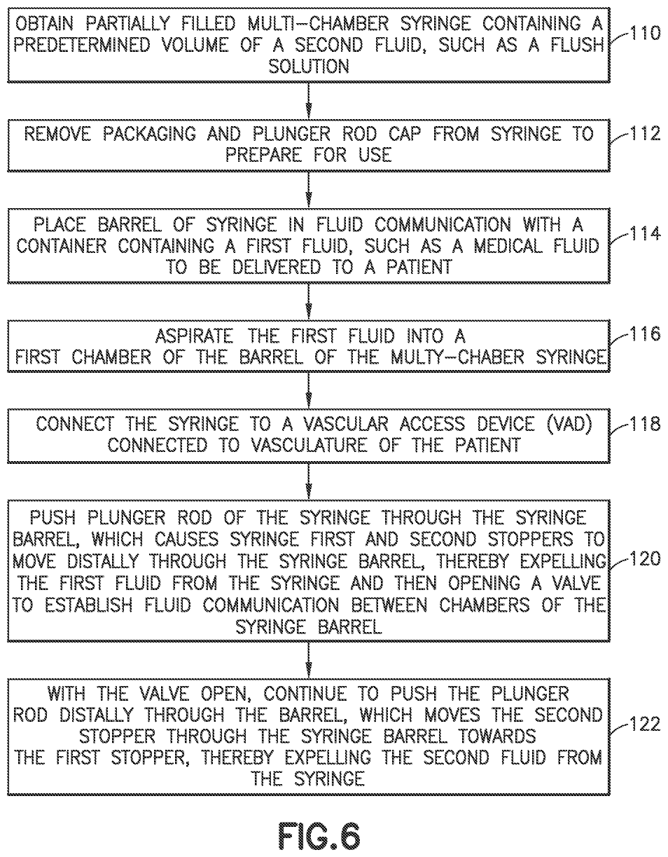

As previously described, the syringe 10 is used for sequential expulsion of fluids, such as expulsion of a medical fluid followed by expulsion of a flush solution, from the syringe 10 to the VAD. A flow chart illustrating steps for a sequential fluid expulsion process using the syringe 10 is shown in FIG. 6.

As shown in FIG. 6, at step 110, a practitioner initially obtains a partially filled syringe 10, in which the second chamber 14 of the syringe 10 is filled with a predetermined volume of a second fluid F2, such as the flush solution. For example, the partially filled syringe 10 can contain about 1 mL to about 20 mL or, preferably, about 5 mL to 10 mL of the flush solution. The syringe 10 in the initial or partially-filled configuration is shown in FIG. 2A. At step 112, the practitioner prepares the syringe 10 for use by, for example, removing any packaging from the syringe 10 and removing the plunger rod cap 58 that holds the plunger rod 40 in place. The practitioner may also move the plunger rod 40 in the distal direction to fully seat the first stopper 26 of the syringe 10 in its distal-most position when the syringe 10 is not initially provided with the first stopper 26 in the distal-most position. At step 114, the practitioner places the syringe barrel 16 in fluid communication with an interior of a container containing the medical fluid to be delivered to the patient. For example, the practitioner may attach a needle (not shown) to the fluid port 22 at the distal end 20 of the syringe barrel 16 and insert the needle into the container, such as a medical vial, containing the medical fluid to be injected to the patient.

At step 116, the practitioner aspirates the first fluid F1 (e.g., the medical fluid) into the first chamber 12 of the barrel 16. For example, the practitioner may grasp the plunger rod 40 and move the plunger rod 40 in the proximal direction, shown by arrow P in FIG. 2B, which moves the stoppers 26, 28 in the proximal direction, thereby aspirating or drawing the first fluid F1 from an interior of the container into the first chamber 12 of the syringe barrel 16, as shown by arrows A1 in FIG. 2A. The syringe 10 is shown in its fully filled configuration in FIG. 2B with the first fluid F1 in the first chamber 12 and the second fluid F2 in the second chamber 14.

Once a dose of the first fluid F1 to be delivered to the patient is drawn into the first chamber 12, at step 118, the practitioner removes the needle from the fluid port 22 of the syringe barrel 16 and connects the fluid port 22 to the VAD. For example, the practitioner may insert a nozzle of the syringe barrel 16 into a corresponding port or valve of the VAD, thereby establishing fluid communication between the syringe barrel 16 and a lumen of the VAD. At step 120, once the syringe 10 is appropriately connected to the VAD, the practitioner grasps the plunger rod 40 and pushes the plunger rod 40 in the distal direction (shown by arrow D in FIG. 2B), which causes the stoppers 26, 28 to move distally through the syringe barrel 16. Distal movement of the first stopper 26 causes the first fluid F1 (e.g., the medical fluid) in the first chamber 12 of the syringe barrel 16 to be expelled from the syringe barrel 16, as shown by arrow A2 in FIG. 2B, to the VAD through the fluid port 22 or nozzle of the syringe barrel 16. The practitioner continues to move the plunger rod 40 in the distal direction until the first stopper 26 is seated in the syringe barrel 16 at its distal-most position. The first stopper 26 is shown in this distal-most position, with the valve in the closed position, in FIG. 2C.

In order to open the valve 60 and establish fluid communication between the second chamber 14 and the first chamber 12, the practitioner continues to move the plunger rod 40 in the distal direction. As previously described, the force required to open the valve 60 may be greater than the force required to move the stoppers 26, 28 through the barrel 16. Accordingly, once the first stopper 26 is in the distal-most position, the practitioner may need to apply additional force to the plunger rod 40 to open the valve 60. Further, as previously described, the distal movement of the plunger rod 40 can be a single continuous stroke in the distal direction performed using one hand (e.g., single-handed operation). As such, the practitioner does not need to perform any other action other than continuing distal movement of the plunger rod 40 to automatically open the valve 60. More specifically, with the first stopper 26 and valve 60 in the distal-most position, the continued distal movement of the plunger rod 40 causes the second stopper 28 to move distally through the barrel 16 towards the first stopper 26. This distal movement of the second stopper 28 increases the fluid pressure in the second chamber 14 towards the activation or opening pressure of the valve 60. Once the fluid pressure in the second chamber 14 reaches or exceeds the activation or opening pressure of the valve 60, the valve 60 opens to establish fluid communication between the second chamber 14 and the first chamber 12 through the channel 30 of the first stopper 26.

At step 122, once fluid communication between the second chamber 14 and the first chamber 12 is established, the practitioner continues to push the plunger rod 40 in the distal direction, which moves the second stopper 28 through the syringe barrel 16 in the distal direction towards the proximal surface 32 of the first stopper 26. As shown by arrows A3 (in FIGS. 2C and 3B), movement of the second stopper 28 towards the first stopper 26 causes the second fluid F2, such as the flush solution, in the second chamber 14 to pass into the channel 30 of the first stopper 26 and into the first chamber 12. The second fluid F2 then passes through the first chamber 12 and is expelled from the syringe barrel 16 to the VAD through the fluid port 22 at the distal end 20 of the syringe barrel 16. The syringe 10 is shown in an end-of-use or final position in FIG. 2D, with the distal surface 34 of the first stopper 26 in contact with or nearly contacting the proximal surface 32 of the first stopper 26.

### Multi-Chamber Syringe Including a Valve With a Sleeve

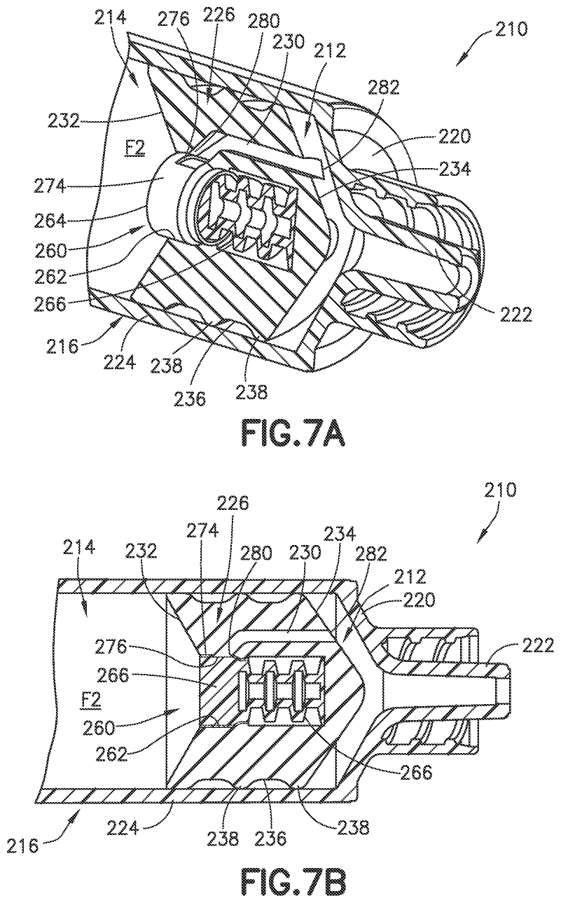

FIGS. 7A and 7B illustrate another example of a multi-chamber syringe 210 for sequential delivery of a first fluid F1, such as a medical fluid, followed by a second fluid F2, such as a flush solution. The syringe 210 includes many of the same components and features of the syringe 10 shown in FIGS. 1A-2D. In particular, the syringe 210 comprises the barrel 216 having an open proximal end, a distal end 220 including a fluid port 222, and a sidewall 224 extending between the proximal end and the distal end 220 of the barrel 216. The syringe 210 further comprises the first stopper 226 and the second stopper (not shown in FIGS. 7A and 7B) slideably positioned in the barrel 216. The first stopper 226 includes a proximal surface 232, distal surface 234, and an outer peripheral surface 236 with annular ribs 238. The first stoppers 226 and the second stopper separate the barrel 216 into chambers 212, 214. Specifically, the syringe 210 includes a first chamber 212 between the distal end 220 of the barrel 216 and the distal surface 234 of the first stopper 226, and a second chamber 214 between the proximal surface 232 of the first stopper 226 and the second stopper. The syringe 210 also includes the valve 260 for controlling fluid flow through the channel 230 of the first stopper 226. As in previous examples, the syringe 210 also includes the plunger rod connected to the second stopper 228 for moving the second stopper through the barrel 216.

The syringe 210 differs from previous examples in the structure of the valve 260. As in previous examples, the valve 260 is a compressible sealing valve including the sealing portion 264 and compressible portion 266, such as an elastomeric bellows. Unlike in previous examples, the valve 260 of the syringe 210 further includes a sleeve 274 around a portion of the valve 260 to separate movable elastomeric portions of the valve 260 from the cavity 262 of the first stopper 226. Separating the movable elastomeric portions of the valve 260 from the first stopper 226 means that the movable portions of the valve 260 do not become stuck to portions of the cavity 262, which may prevent the valve 260 from opening and closing in an expected manner. Accordingly, the sleeve 274 reduces or eliminates effects of friction or stiction between the valve 260 and the first stopper 226 in a manner similar to the lubricant coating of previous examples.

The sleeve 274 is formed from a different material than the valve 260 and/or the first stopper 226. For example, the sleeve 274 can be formed from a rigid thermoplastic material, such as polyester, polycarbonate, polypropylene, polyethylene, polyethylene terephthalate, or acrylonitrile butadiene styrene. In contrast, the first stopper 226 and valve 260 are formed from a thermoplastic elastomer, such as natural rubber (e.g., isoprene). In some examples, the sleeve 274 is an annular sleeve surrounding the sealing portion 264 of the valve 260. The sleeve 274 can include an opening 276 positioned proximate to the inlet 280 of the channel 230 so that the sealing portion 264 of the valve 260 contacts and seals the inlet 280 when the valve 260 is closed. The sleeve 274 is configured to move away from the inlet 280 along with the sealing portion 264 of the valve 260 as the valve 260 moves to the open position.

The syringe 210 operates in the same manner as previous examples. Specifically, once the syringe 210 is fully filled with the first fluid F1 in the first chamber 212 and the second fluid F2 in the second chamber 214, the fluids F1, F2 are expelled from the syringe 210 by moving the plunger rod in the distal direction. Distal movement of the plunger rod causes the first stopper 226 and the second stopper to move distally through the syringe barrel 216. As in previous examples, the first fluid F1 is expelled from the first chamber 212 through the fluid port 222 of the barrel 216 until the first stopper 226 is seated in its distal-most position against the distal end 220 of the barrel 216, as shown in FIG. 9A.

Continued distal movement of the plunger rod (not shown in FIGS. 7A and 7B) causes the second stopper to move toward the first stopper 226, which increases the fluid pressure in the second chamber 214 until the valve 260 opens. Once the valve 260 opens, the second fluid F2 moves through the channel 230 of the first stopper 226, into the first chamber 212, and then from the barrel 216 through the fluid port 222. Accordingly, the syringe 210 provides for sequential expulsion of the first fluid F1 followed by the second fluid F2 with a single continuous movement of the plunger rod in the distal direction. The practitioner can perform the single continuous movement with one hand meaning that the syringe 210 is capable of single-hand operation.

While examples of the multi-chamber syringes and methods of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A multi-chamber syringe for sequential expulsion of at least a first fluid followed by a second fluid, the syringe comprising:
a barrel comprising a proximal end, a distal end comprising a fluid port for expulsion of the first fluid and the second fluid from the barrel, and a sidewall extending between the proximal end and the distal end of the barrel;
a first stopper comprising at least one channel, the first stopper being slidably positioned in the barrel with a first chamber configured to contain the first fluid between the distal end of the barrel and the first stopper;
a second stopper slidably positioned in the barrel proximal to the first stopper with a second chamber configured to contain the second fluid between the first stopper and the second stopper; and
a valve which transitions between (i) a closed position, where fluid flow through the at least one channel of the first stopper is prevented, when a fluid pressure in the second chamber is below a predetermined opening pressure, and (ii) an open position when fluid pressure in the second chamber is greater than or equal to the predetermined opening pressure, thereby establishing fluid communication between the second chamber and the first chamber through the at least one channel of the first stopper.

2. The syringe of claim 1, wherein movement of the second stopper through the barrel in a proximal direction causes the first stopper to move in the proximal direction to aspirate the first fluid into the first chamber through the fluid port of the barrel.

3. The syringe of claim 1, with the first stopper in a distal-most position, movement of the second stopper towards the first stopper causes fluid pressure in the second chamber to increase towards the predetermined opening pressure of the valve.

4. The syringe of claim 1, wherein the fluid port comprises a nozzle having a minimum cross-sectional area that is smaller than a cross-sectional area of an outlet of the at least one channel of the first stopper.

5. The syringe of claim 1, wherein the predetermined opening pressure of the valve is at least 30 psi.

6. The syringe of claim 1, wherein the valve comprises a single use valve that transitions from the closed position to the open position when the fluid pressure of the second chamber is equal to or greater than the opening pressure of the valve, and which, once open, does not return to the closed position.

7. The syringe of claim 1, wherein the valve comprises a one way check valve comprising at least one of a ball valve, compression sealing valve, diaphragm valve, tilting disc valve, clapper valve, or duckbill valve.

8. The syringe of claim 1, wherein the valve comprises a compressible seal that covers an inlet of the at least one channel, when the valve is in the closed position, and which compresses to uncover the inlet when the fluid pressure of the second chamber is equal to or greater than the predetermined opening pressure of the valve.

9. The syringe of claim 1, wherein the first stopper comprises a cavity extending inwardly from a proximal surface of the first stopper, and wherein the valve is seated in the cavity.

10. The syringe of claim 9, wherein an inlet of the at least one channel extends through an inner surface of the cavity,
wherein an outlet of the at least one channel extends through a distal surface of the first stopper, and
wherein the valve comprises a sealing portion positioned to cover the inlet of the at least one channel when the valve is closed, and a compressible portion, which compresses as the fluid pressure increases, causing the sealing portion of the valve to retract into the cavity away from the inlet of the at least one channel.

11. The syringe of claim 9, wherein the compressible portion comprises an elastomeric bellows comprising at least one radially extending flange sized to contact the inner surface of the cavity to support the valve.

12. The syringe of claim 9, wherein the first stopper comprises a protruding lip extending into the cavity that engages the valve, when the valve is in the open position, preventing the valve from closing when the fluid pressure in the second chamber decreases below the opening pressure of the valve.

13. The syringe of claim 9, wherein an outer surface of the valve and/or an inner surface of the cavity comprises a lubricant coating so that the valve slides through the cavity as the fluid pressure of the second chamber increases.

14. The syringe of claim 9, further comprising an annular sleeve around at least a portion of the valve separating elastomeric portions of the valve from elastomeric portions of the first stopper.

15. The syringe of claim 1, further comprising a plunger rod connected to the second stopper for moving the second stopper through the barrel.

16. The syringe of claim 15, wherein the syringe is configured such that the first fluid followed by the second fluid are expelled from the syringe barrel from a single continuous advancement of the plunger rod distally into the syringe barrel, and/or
wherein the syringe is configured such that the first fluid followed by the second fluid are expelled from the syringe barrel by single-handed operation of the plunger rod.

17. The syringe of claim 15, wherein the plunger rod is fixedly connected to the second stopper and the plunger rod and/or second stopper are free from connections to the first stopper.

18. A pre-filled flushing syringe, comprising:
the syringe of claim 1; and
a predetermined volume of the second fluid disposed in the second chamber of the pre-filled syringe,
wherein the pre-filled syringe is provided with the first chamber fluidly isolated from the second chamber, thereby containing the predetermined volume of the second fluid within the pre-filled syringe.

19. A method for sequential expulsion of fluids from the syringe of claim 1, the method comprising:
moving a plunger rod fixedly connected to the second stopper of the syringe in a proximal direction, which moves the first stopper of the syringe in the proximal direction, to aspirate the first fluid into the first chamber of the syringe barrel; and
once a dose of the first fluid is in the first chamber, moving the plunger rod in the distal direction, as a single continuous stroke, thereby causing the first fluid followed by the second fluid to be expelled from the fluid port of the syringe barrel.

20. The method of claim 19, wherein the single continuous stroke of the plunger rod is performed as a single-hand operation.
